(19) United States
(12) Patent Application Publication
(10) Pub. No.: US 2007/0283959 A1
(43) Pub. Date: Dec. 13, 2007

(54) AIRCRAFT OXYGEN SUPPLY UNIT

(75) Inventor: Thomas Rassloff, Lubeck (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: DRAGER AEROSPACE GMBH, Lubeck (DE)

(21) Appl. No.: 11/752,491

(22) Filed: May 23, 2007

(30) Foreign Application Priority Data
May 23, 2006 (DE) 10 2006 024 052.9

(51) Int. Cl. A61M 16/00; A62B 18/02

(52) U.S. Cl. 128/204.18; 128/205.25

(57) ABSTRACT

An oxygen supply unit, particularly for use in aircraft, is provided which is arranged in a portable transport container (16) having at least one oxygen storage device (2, 2′, 2″) and a breathing requirement regulator (14, 14′14″) in fluid connection to the storage devices via a flexible tubing conduit (12, 12′, 12″), to which an oxygen mask (36, 36′, 36″) may be connected at an exit side of the conduit.

### BACKGROUND OF THE INVENTION

The invention relates to an oxygen supply unit, particularly for aircraft.

Aircraft with a pressurized interior comprise oxygen supply systems, which ensure an adequate oxygen supply of the pressurized cabin under normal flight conditions. Apart from this, these aircraft are equipped with emergency oxygen supply installations, by which the occupants of the aircraft may be supplied with oxygen as an emergency given a pressure drop in the cabin, in particular at a high altitude.

Apart from this emergency pressure compensation between cabin- and atmospheric pressure, situations however occur in which an intended pressure compensation between the pressurized cabin and the surroundings of the aircraft is carried out. Examples of this are when loads and/or persons are dropped off at middle or high altitude within the framework of relief operations or military operations. During the phases of the flight in which the cabin inner pressure is compensated to the atmospheric outer pressure, the occupants of the aircraft must be respirated with oxygen or with air enriched with oxygen. Since the emergency supply systems must be carried along as a precautionary measure for emergency situations, additional oxygen supply installations are necessary with the deployment profile described above, which may supply the aircraft occupants with oxygen, as the case may be, over a longer period of time.

Known installations of this type comprise a large, high-pressure oxygen tank, or several medium-sized such tanks, which are connected together into a supply unit. The oxygen which is stored there is led to the occupants of the aircraft, who may be in the cockpit or in the freight space of the aircraft, for breathing. Thus, the aircraft occupants are supplied from a common oxygen storage device or oxygen storage system. The high-pressure oxygen tank or tanks are arranged on pallets and, when required, are brought into the aircraft. There, the supply installation is connected to the on-board energy supply systems. For this, suitable connections and connection conduits must be provided in the aircraft. Furthermore, the aircraft with which the outlined flights missions are carried out are equipped to the extent that, in the case of a leakage of the high-pressure oxygen tank in the aircraft interior, bleed conduits are provided which lead to the outboard of the aircraft via openings on the outer wall.

### BRIEF SUMMARY OF THE INVENTION

Against this background, it is an object of the invention to provide an oxygen supply unit, particularly for aircraft, which ensures a reliable oxygen supply, and thereby is simple to handle and may be applied in a flexible and comprehensive manner.

According to the invention, an aircraft oxygen supply unit is provided, which is arranged in a portable transport container. The aircraft oxygen supply unit (hereinafter sometimes simply referred to as “oxygen supply unit,” it being understood that the unit is particularly designed for aircraft use, but could be used in other high altitude environments or wherever emergency oxygen is required) comprises at least one oxygen storage device and a breathing requirement regulator in fluid connection therewith via a flexible tubing conduit. On the exit side, an oxygen mask may be connected to this breathing requirement regulator.

Ideally, the oxygen supply unit is designed such that it does not need to be connected to the supply- and/or conduit system of the aircraft. The oxygen supply unit is thus independent of all devices incorporated in the aircraft. In contrast to known oxygen supply installations of this type, no conversion measures are necessary for the aircraft, so that an existing airworthiness certificate of the aircraft is not compromised by the application of the oxygen supply unit according to the invention, and so that no changes on the aircraft need to be carried out for the operation, which would require notification with regard to the airworthiness certificate. Accordingly, any conversion costs on the applied aircraft, as well as all further additional costs associated with the aircraft, are dispensed with on operating the aircraft oxygen supply unit according to the invention.

The oxygen supply unit according to the invention is advantageously dimensioned such that it may be brought into the inside of the aircraft without additional aids, such as hoisting devices or transport devices. In this manner, almost every aircraft, which is suitable for the application scenarios described above, may be equipped with the invention in the shortest of times for such applications, without any special infrastructure. For example, an aircraft may also be equipped with the oxygen supply unit at those take-off and landing locations which do not have any transport- and hoisting devices. A further advantage is the fact that with the oxygen supply unit according to the invention, the maintenance and overhauling is considerably simplified compared to the previously known installations of this type. Thus with regard to this, no work whatsoever is required in or on the aircraft. Instead, the aircraft oxygen supply unit may, for example, be easily brought into a workshop without the use of aids.

At least all essential components of the oxygen supply unit, i.e., the oxygen storage device or devices, flexible supply tubing, fittings and the breathing requirement regulator, may be stored in the transport container, protected from environmental influences and possible damage during the transport and storage. If the oxygen supply unit is applied in an aircraft, the flexible supply tubing with the breathing requirement regulator connected thereto, is applied outside the transport container to the user or users of the oxygen supply unit, and the oxygen storage devices may continue to be kept in the transport container in a protected manner.

Preferably, a compressed gas container serves as an oxygen storage device, but alternatively, it is also conceivable to provide a chemical oxygen generator as an oxygen storage device, with which oxygen which is bonded to at least one chemical component, is released by way of chemical reaction.

The flexible tubing conduit at whose free end the breathing requirement regulator is arranged, connects to the oxygen storage device. An oxygen mask is provided at the exit side of this breathing requirement regulator. The oxygen mask may be in fluid connection therewith the breathing requirement regulator in a fixed manner, but it is beneficial to provide a coupling system with which, when required, an oxygen mask may be connected to the requirement regulator. This has the advantage that the aircraft occupants may connect individual masks to the oxygen supply unit, which are adapted to their personal facial physiognomy and are accordingly comfortable when worn.

The breathing requirement regulator may be designed such that it feeds a constant oxygen flow to the breathing mask. The breathing requirement regulator, however, is advantageously designed such that it provides the breathing mask with a quantity of oxygen which is directed to requirement and is adapted to the flight situation. Thus, the oxygen quantity may be adapted advantageously to the increased oxygen requirement at a greater altitude, by way of the breathing requirement regulator.

The oxygen supply unit is preferably designed for the supply of oxygen to several aircraft occupants. In each case, an individual oxygen supply, with an oxygen storage device and with breathing requirement regulator in fluid connection therewith, is provided for each aircraft occupant to be supplied. The concept behind this arrangement is to minimize the effects of a possible malfunction of the oxygen supply unit to the extent that not all aircraft occupants are affected by a malfunction of the oxygen supply unit. Each of the aircraft occupants is supplied with the breathing gas from an oxygen supply which is separate from the other aircraft occupants. A failure or malfunctioning of this one oxygen supply, in the worst case scenario, results in the user concerned not being fed with oxygen via this oxygen supply, but the supply to the other aircraft occupants continuing to be effected via their independent oxygen supplies. With this arrangement, several oxygen storage devices may be arranged in the aircraft oxygen supply unit in a transport container, wherein a breathing requirement regulator and, as the case may be, an oxygen mask are connected to said oxygen storage devices in each case via a flexible tubing conduit, in the manner described above.

The oxygen storage device advantageously comprises at least two pressurized oxygen bottles. Thus the oxygen may be transported and stored in commercially available, standardized pressurized gas bottles, which are grouped together into bundles of bottles. It is possible by way of this, to adapt the storage volume of the oxygen storage device in a simple manner to the application requirements during a flight, i.e., to the application duration and the number of persons to be supplied.

When a pressurized oxygen bottle of a user has been emptied with the application of the aircraft oxygen supply unit according to the invention, the user connects a further filled pressurized oxygen bottle to his oxygen mask. For this, a switch-over valve is usefully provided, to which the pressurized oxygen bottles are connected. The switch-over valve may thereby be designed such that it is switched over manually by the user of the oxygen supply. Preferably, however, an automatic switch-over is provided on the switch-over valve, on reaching a certain pressure level of an emptied or almost emptied pressurized oxygen bottle.

A further advantageous design of the aircraft oxygen supply unit according to the invention envisages means for the emergency closure of the oxygen storage device. These means may be useful when, for example in the somewhat long flexible tubing connection between the oxygen storage device and the breathing requirement regulator, oxygen flows into the inside of the aircraft in an uncontrolled manner as a result of damage to the flexible tubing.

In a useful further embodiment of the invention, a second switch-over valve is arranged on the entry side of the breathing requirement regulator. The breathing requirement regulator is preferably worn in the direct vicinity of the body of the user of the oxygen mask. If a switch-over valve is arranged in the entry side of this breathing requirement regulator, then this valve may be easily reached by the user of the oxygen mask. A further oxygen storage device, preferably a pressurized oxygen bottle, is connected to the switch-over valve. In this manner, it is possible for the user of the oxygen supply to switch over from one oxygen storage device to the other in a direct manner, without previously having to look for transport containers which may be distanced further away, in order to carry out the switch-over.

Such a switch-over from one oxygen storage device to another oxygen storage device may be required, for example when the user of the oxygen supply wishes to move outside the actual reach of the aircraft oxygen supply unit, which in the normal case is given by the length of the flexible tubing connection between his oxygen storage device arranged in the transport container and the oxygen mask. For this purpose, a releasable coupling for coupling and decoupling of the flexible tubing connection to the oxygen storage device, arranged advantageously in the transport container, and a further portable oxygen storage device are provided on the second switch-over device at the entry side of the breathing requirement regulator. This permits the user of the oxygen supply to couple a portable oxygen storage device, for example a small pressurized oxygen bottle which may be carried on the body, to the switch-over valve, and to decouple the flexible tubing connection, existing at the oxygen storage device in the transport container, from the switch-over valve. The user may now move within the aircraft, being supplied with oxygen, independently of the length of the flexible tubing.

The transport containers of the aircraft oxygen supply unit in a preferred embodiment are designed as essentially rectangular, stackable metal containers. Preferably, metal cases, for example commercially available aluminum cases or instead other alloy metal boxes, which may be closed with a lid, are provided as transport containers, and these beneficially have carrier grips, so that the transport containers may be easily carried by one or more persons into or out of the aircraft.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective view of an aircraft oxygen supply unit according to one embodiment of the invention;

FIG. 2 is a schematic representation of the functional construction of the aircraft oxygen supply unit according to FIG. 1;

FIG. 3 is a perspective view of an aircraft oxygen supply unit according to another embodiment of the invention, with an additional portable oxygen storage device;

FIG. 4 is a schematic representation of the functional construction of the aircraft oxygen supply unit according to FIG. 3; and

FIG. 5 is a schematic representation of the functional construction of an aircraft oxygen supply unit for supplying three aircraft occupants with oxygen.

### DETAILED DESCRIPTION OF THE INVENTION

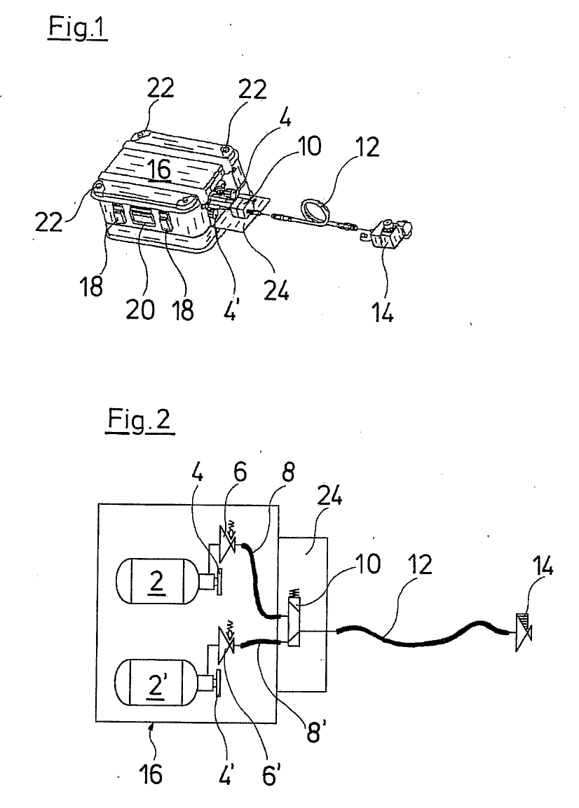

The aircraft oxygen supply unit represented by way of FIGS. 1 and 2 comprises two pressurized oxygen bottles 2 and 2′, to whose exits, in each case a shut-off valve 4 and 4′ as well as a pressure reducer 6 and 6′ are arranged respectively, as is usual for gas bottles. In each case, a flexible connection tubing 8 and 8′ connects to the pressure reducers 6 and 6′, wherein the two flexible connection tubings 8 and 8′ at their other ends are connected to two entries of a switch-over valve 10. A flexible supply tubing 12 is connected to the switch-over valve 10 at the exit side of this, and the other end of this flexible tubing is connected to a breathing requirement regulator 14. An oxygen mask which is not represented in the FIGS. 1 and 2, may be connected to this breathing requirement regulator 14.

FIG. 1 shows that the pressurized oxygen bottles 2 and 2′ together with the shut-off valves 4 and 4′ as well as the pressure reducers 6 and 6′ are arranged in a transport container 16. The transport container 16 is designed as an essentially rectangular metal case, in which all components of the oxygen supply may be accommodated and are protected, for the transport and for storage. The transport container 16 is opened via a lid which forms the upper side of the transport container. This lid may be folded open on the transport container via hinges arranged on one side. The hinges may not be seen in the Figs. The lid may however also be arranged on the transport container in a manner such that it may be lifted up. The lid is closed by two snap closures 18 arranged on a side surface of the transport container, as for example are known from traveler's cases and executive cases. Of course other closure systems are also conceivable as closures. For example the transport container may also be closed by clamp closures and buckle closures. A carrier grip 20 is attached between the closures 18, as is usual with cases, with which a person may carry the oxygen supply unit.

In each case a cylindrical shaped peg-like lug 22 extends upwards from the surface of the lid in a free-standing manner, in the region of all four corners of the lid. These lugs 22 engage into correspondingly shaped recesses on the base of the upper transport container 16 placed thereon, when stacking several transport containers 16 above one another. A fixation against dislocation of the transport containers is formed in this manner.

The pressurized oxygen bottles 2 and 2′ are aligned in the transport containers 16 such that their fittings face a further flap 24 on a side wall of the case. By way of opening the flap 24, it is then possible to remove the switch-over valve 10 with the flexible supply tubing 12, which is arranged thereon and to which the breathing requirement regulator 14 connects, from the transport container 16, and to provide it for the oxygen supply via an oxygen mask which may be connected thereto. An access to the inside of the transport container 16 is thus possible via the flap 24, without having to open the actual lid of the transport container 16. This firstly permits several transport containers 16 with the oxygen supply devices arranged therein, to be arranged stacked over one another in an aircraft, and an oxygen supply to several aircraft occupants via the respective flaps 24.

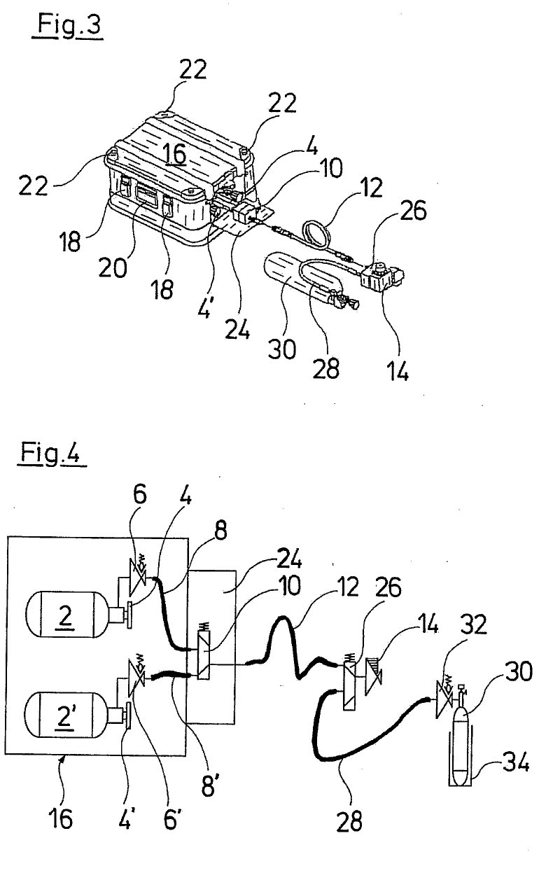

FIGS. 3 and 4 show a further embodiment of the aircraft oxygen supply unit which essentially corresponds to that shown in FIGS. 1 and 2. The oxygen supply unit shown here also comprises two pressurized gas storage devices 2 and 2′ which are arranged in a transport container 16. In each case a shut-off valve 4 and 4′ with a pressure reducer 6 and 6′ arranged downstream, is arranged at the gas exit of the pressurized gas tanks 2 and 2′ respectively, and flexible tubing connections 8 and 8′ departing from the pressure reducers 6 and 6′ respectively, run into a switch-over valve 10 on the entry side.

A flexible supply tubing 12 connects to the switch-over valve 10 at the exit side of this, and this flexible tubing at its other end is connected to an entry of a further switch-over valve 26. The switch-over valve 26 comprise a further entry, to which a further pressurized oxygen bottle 30 with a pressure reducer 32 connected upstream, is connected via a flexible supply tubing 28. The pressurized oxygen bottle 30 is significantly smaller than the pressurized oxygen bottles 2 and 2′, and is received by a carrier bag 34. The pressurized oxygen bottle 30 may be worn on the body of a user of the oxygen supply unit by way of the carrier bag 34. A breathing requirement regulator 14 is connected to the switch-over valve 26 at its exit side, and an oxygen mask (not represented in FIGS. 3 and 4) may be connected to the breathing requirement regulator at the exit side.

The switch-over valve 26 is designed such that a flow path to the breathing requirement regulator 14 may be switched selectively by way of a manual switch-over from the flexible supply tubing 12 in fluid connection with the pressurized gas bottles 2 and 2′ or the flexible supply tubing 28 in fluid connection with the portable pressurized gas bottle 30. It is thus possible for the user of the oxygen supply unit at a certain location, to be supplied with oxygen by way of the supply conduit 12 from the pressurized oxygen bottles 2 and 2′, but when required the user may connect the flexible tubing 28 to the switch-over valve 26, and then create a conducting connection to the pressurized oxygen bottle 30 by way of switching over this switch-over valve 26. The user of the oxygen supply may now be supplied with the oxygen from the pressurized oxygen bottle 30. The user is in the position of separating the supply conduit 12 from the switch-over valve 26, and may move within the aircraft in a mobile manner. In this way, the user's scope of movement within the aircraft is no longer limited by the length of the conduit connection between the transport container 16 and his oxygen mask.

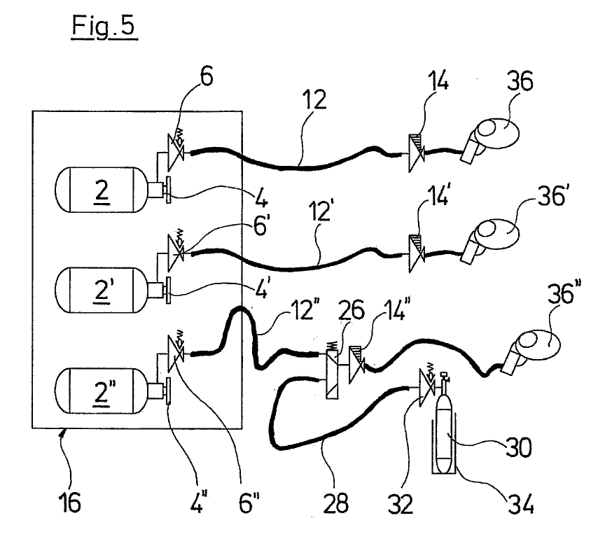

Another aircraft oxygen supply unit is sketched in FIG. 5, with which three aircraft occupants may be supplied with oxygen. For this, three pressurized oxygen bottles 2, 2′ and 2″ are arranged in a transport container 16, which in the described manner are each in fluid connection with breathing requirement regulators 14, 14′ and 14″. In each case, an oxygen mask 36, 36′, and 36″ is connected to the exit of these breathing requirement regulators 14, 14′ and 14″. Thus three oxygen supply systems which are independent of one another are arranged in the transport container 16, with which three persons may be supplied with oxygen in an aircraft. A first person may inhale oxygen from a pressurized oxygen bottle 2 via the oxygen mask 36, the breathing requirement regulator 14 connected upstream of this oxygen mask 36, the flexible supply tubing 12 connected to the regulator, and the pressure reducer 6.

The same applies to two further persons, wherein the oxygen mask 36′ in the same manner is in fluid connection with a pressurized oxygen bottle 2′ via a breathing requirement regulator 14′, a flexible supply tubing 12′ and via a pressure reducer 6′. The user of the oxygen mask 36″ may inhale oxygen from the pressurized oxygen bottle 2″ as well as from a pressurized oxygen bottle 30 located in a carrier bag 34. For this, a switch-over valve 26 is arranged on the entry side of the breathing requirement regulator 14″ connected upstream of the oxygen mask 36″. The pressurized oxygen bottle 2″ via the flexible supply tubing 28″, or the pressurized oxygen bottle 30 via the flexible supply tubing 12″, may be selectively connected to this switch-over valve 26. Thus, the user of the oxygen mask 36′,′ on the one hand, may be supplied with oxygen at a fixed location from the pressurized oxygen bottle 2″, and on the other hand, for leaving this location, he may connect the pressurized oxygen bottle 30 to the breathing requirement regulator 14″ and then breath oxygen from the portable pressurized oxygen bottle 30.

The number of pressurized oxygen bottles 2, 2′ and 2″ represented in FIG. 5 in a transport container 16, with the oxygen masks 36, 36′ and 36″ connected to the bottles, is purely an example. Thus the aircraft oxygen supply unit may for example be designed for supplying more or fewer persons. It is also possible in each case for several pressurized oxygen bottles to be provided for the oxygen supply of one person.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. An oxygen supply unit, comprising a portable transport container (16), at least one oxygen storage device (2, 2′, 2″) arranged in the portable transport container (16), a breathing requirement regulator (14, 14′, 14″) in fluid connection with the at least one oxygen storage device via a flexible tubing conduit (12, 12′, 12″), to which regulator an oxygen mask (36, 36′, 36″) is connectable at an exit side of the regulator.

2. The oxygen supply unit according to claim 1, designed for the oxygen supply of several aircraft occupants, such that in each case an independent oxygen supply provided with one oxygen storage device (2, 2′, 2″) and with a breathing requirement regulator (14, 14′, 14″) in fluid connection therewith, is provided for each aircraft occupant to be supplied.

3. The oxygen supply unit according to claim 1, wherein the oxygen storage device (2, 2′, 2″) comprises at least two pressurized oxygen bottles (2, 2′, 2″).

4. The oxygen supply unit according to claim 3, further comprising a switch-over valve (10) to which the pressurized oxygen bottles (2, 2′, 2″) are connected.

5. The oxygen supply unit according to claim 1, further comprising an emergency closure for the oxygen storage devices (2, 2′, 2″).

6. The oxygen supply unit according to claim 1, further comprising a second switch-over valve (26) arranged on an entry side of the breathing requirement regulator (14, 14′, 14″)

7. The oxygen supply unit according to claim 6, wherein the second switch-over valve (26) comprises a releasable coupling for coupling and decoupling the flexible tubing connection (12, 12′, 12″) to the oxygen storage device (2, 2′, 2″) and to a further portable oxygen storage device (30).

8. The oxygen supply unit according to claim 1, wherein the transport container (16) is designed as an essentially rectangular, preferably stackable metal container.

9. The oxygen supply unit according to claim 8, wherein the transport container (16) is stackable with metal container.

10. The oxygen supply unit according to claim 1, wherein the unit is adapted for use in aircraft.
